(19) United States
(12) Patent Application Publication
(10) Pub. No.: US 2024/0181454 A1
(43) Pub. Date: Jun. 6, 2024

(54) CENTRIFUGE APHERETIC SYSTEM

(71) Applicant: ASTRIN BIOSCIENCES, INC., Excelsior, MN (US)

(72) Inventors: Jiarong HONG, Excelsior, MN (US); Mahdi AHMADI, Excelsior, MN (US); Kaylee SMITH, Excelsior, MN (US); Jayant PARTHASARATHY, Excelsior, MN (US)

(73) Assignee: ASTRIN BIOSCIENCES, INC., Excelsior, MN (US)

(21) Appl. No.: 18/062,311

(22) Filed: Dec. 6, 2022

(51) Int. Cl. B01L 3/00; G01N 35/00

(52) U.S. Cl. CPC B01L 3/502753; G01N 35/0099; B01L 2200/04; B01L 2200/0652; B01L 2300/0654; B01L 2400/0409

(57) ABSTRACT

The present embodiments relate to an apheretic system incorporating one or more centrifuge devices. A biological fluid containing cells (or another fluid) can be obtained and directed to a centrifuge device to separate particles by a particle size and/or a flow rate. The centrifuge device can include a center-axis centrifuge, an off-center centrifuge, and/or a hydro-cyclone centrifuge device. The separated particles can be directed/drawn into either an outlet to a return or into a collection container for further testing.

### TECHNICAL FIELD

The methods and compositions described here relate to the field of apheretic systems. More particularly, embodiments relate to an apheretic system incorporating one or more centrifuge devices to separate and isolate certain cell types.

### BACKGROUND

A centrifuge can include a device that uses centrifugal force to separate components or particles in a fluid. For example, a centrifuge-base method can be used to separate blood components in a biological fluid containing cells, such as red blood cells, nucleated cells, and plasma. Different methods for separating particles can be used based on the target component. Further, both continuous and intermittent centrifugation can be used.

Centrifugation techniques can either separate plasma from cells, platelets, or cellular components. Regardless of application, these technologies can use a large bowl or a similar object to isolate particles by centrifugation.

### SUMMARY

The present embodiments relate to an apheretic biopsy system incorporating one or more centrifuge devices. In one embodiment, a centrifugal apheretic system is described. The centrifugal apheretic system can include an inlet configured to obtain a biological fluid containing cells. The centrifugal apheretic system can also include a collection container configured to collect the biological fluid containing cells from the inlet.

The centrifugal apheretic system can also include a centrifuge device connected to the collection container. The centrifuge device can be configured to rotate about an axis, causing particles in the biological fluid containing cells to separate in the collection container by a particle size and/or a particle flow rate. The centrifugal apheretic system can also include an outlet configured to direct a first portion of the separated particles in the biological fluid containing cells from the collection container to a first channel of the outlet and direct a second portion of the separated particles in the biological fluid containing cells from the collection container to a second channel of the outlet.

In some instances, the centrifugal apheretic system can also include a robotic arm connected to the inlet and a dual needle connected to the robotic arm. The robotic arm can be configured to move the dual needle into the collection container, wherein a first needle of the dual needle is configured to provide the biological fluid containing cells to the collection container, and a second needle of the dual needle is configured to direct the separated particles from the collection container to the outlet.

In some instances, the centrifuge device is a center axis centrifuge device configured to rotate about a central axis, and wherein the collection container is disposed at a periphery of the center axis centrifuge device.

In some instances, the centrifuge device is a spinning centrifuge device configured to surround the collection container and rotate the collection container about the central axis.

In some instances, the centrifuge device is an off-center axis centrifuge device configured to rotate offset relative to the central axis.

In some instances, the system also includes at least two off-center axis centrifuge devices connected to the inlet in parallel, wherein each of the at least two off-center axis centrifuge devices comprise collection containers and each collection container is configured to receive part of the biological fluid containing cells.

In some instances, the centrifugal apheretic system can also include a motorized valve connected to the outlet, wherein the motorized valve is configured to open to either the first channel to direct the first portion of the separated particles to the first channel or the second channel to direct the second portion of the separated particles to the second channel.

In some instances, the centrifugal apheretic system can also include an image sensor connected to the motorized valve and configured to capture an image of the separated particles of the biological fluid containing cells and a computing node electrically connected to the sensor. The computing node can include a processor and a memory. The memory can comprise instructions that are configured to cause the processor to: obtain the image from the image sensor. The memory can also cause the processor to derive a parameter of at least a portion of separated particles depicted in the image. The memory can also cause the processor to determine whether the separated particles depicted in the image are part of the first portion or the second portion of the biological fluid containing cells based on the derived parameter. The memory can also cause the processor to cause the motorized valve connected to the outlet to open either the first channel or the second channel based on whether the separated particles included in the image are part of the first portion or the second portion of the biological fluid containing cells.

In some instances, the biological fluid containing cells comprises a stream of blood cells, and wherein the memory of the computing node comprises instructions that are further configured to cause the processor to: determine a color of at least the portion of the separated particles depicted in the image and derive a hematocrit level of the at least the portion of the separated particles depicted in the image based on the determined color.

In some instances, the first channel is configured to direct the first portion of the separated particles to another container for further testing, and the second channel is configured to direct the second portion of the separated particles to a patient via a return line.

In another example embodiment, a method is provided. The method can include obtaining, at an inlet, a biological fluid containing cells. The biological fluid containing cells can be directed from the inlet to a collection container connected to a centrifuge device.

The method can also include causing rotation of the centrifuge device about an axis to separate particles in the biological fluid containing cells in the collection container. The method can also include drawing a first portion of the separated particles in the biological fluid containing cells from the collection container to a first channel of the outlet. The method can also include drawing a second portion of the separated particles in the biological fluid containing cells from the collection container to a second channel of the outlet.

In some instances, the method can also include pressurizing, by a pump, the biological fluid containing cells in the inlet, wherein the biological fluid containing cells is obtained at the inlet from a patient, and wherein the second portion of the separated particles in the biological fluid containing cells is directed from the second channel to the patient.

In some instances, the method can also include opening, by a motorized valve connected to the outlet, either the first channel to direct the first portion of the separated particles to the first channel or the second channel to direct the second portion of the separated particles to the second channel.

In some instances, the method can also include capturing, by an image sensor connected to the outlet, an image of the separated particles of the biological fluid containing cells, obtaining, by a computing node, the image from the image sensor, deriving, by the computing node, a parameter of at least a portion of separated particles depicted in the image, determining, by the computing node, whether the separated particles depicted in the image are part of the first portion or the second portion of the biological fluid containing cells based on the derived parameter, and causing, by the computing node, the motorized valve connected to the outlet to open either the first channel or the second channel based on whether the separated particles included in the image are part of the first portion or the second portion of the biological fluid containing cells.

In some instances, the method can also include moving, by a robotic arm connected to the inlet and to a dual needle, the dual needle into the collection container, wherein a first needle of the dual needle is configured to provide the biological fluid containing cells to the collection container and a second needle of the dual needle is configured to direct the separated particles from the collection container to the outlet.

In some instances, the method can also include disposing, by an injector, an anti-coagulant into the biological fluid containing cells in the inlet.

In another example embodiment, a system is provided. The system can include an inlet configured to obtain a biological fluid containing cells. The system can also include a collection container configured to collect the biological fluid containing cells from the inlet. The system can also include a centrifuge device connected to the collection container, wherein the centrifuge device is configured to rotate about an axis, causing particles in the biological fluid containing cells to separate in the collection container.

The system can also include an outlet comprising a first channel and a second channel. The system can also include a motorized valve configured to open the first channel and the second channel. The system can also include an image sensor connected to the motorized valve and configured to capture an image of the separated particles of the biological fluid containing cells.

The system can also include a computing node electrically connected to the sensor, wherein the computing node comprises a processor and a memory. The memory can include instructions that are configured to cause the processor to obtain the image from the image sensor. The memory can further cause the processor to derive a particle size of at least a portion of separated particles depicted in the image. The memory can further cause the processor to determine whether the separated particles depicted in the image are part of the first portion or the second portion of the biological fluid containing cells based on the derived particle sizes. The memory can further cause the processor to cause the motorized valve connected to the outlet to open either the first channel or the second channel based on whether the separated particles included in the image are part of the first portion or the second portion of the biological fluid containing cells.

In some instance, the outlet is configured to direct a first portion of the separated particles in the biological fluid containing cells from the collection container to the first channel of the outlet and direct a second portion of the separated particles in the biological fluid containing cells from the collection container to the second channel of the outlet.

In some instances, the system can also include a robotic arm connected to the inlet and a dual needle connected to the robotic arm. The robotic arm can be configured to move the dual needle into the collection container, wherein a first needle of the dual needle is configured to provide the biological fluid containing cells to the collection container, and a second needle of the dual needle is configured to direct the separated particles from the collection container to the outlet.

In some instances, the centrifuge device is a center axis centrifuge device configured to rotate about a central axis, and wherein the collection container is disposed at a periphery of the center axis centrifuge device.

### BRIEF DESCRIPTION OF THE DRAWINGS

Embodiments of the present invention are illustrated by way of example and not limitation in the figures of the accompanying drawings, in which like references indicate similar elements and in which:

FIG. 1 illustrates a robotic-assisted centrifuge design according to an embodiment.

FIGS. 2A-2C illustrate an example center-axis spinning centrifuge according to an embodiment.

FIG. 3 illustrates an example off-center active spinning centrifuge according to an embodiment.

FIG. 4 is an example flow process for moving a motorized valve based on a determined layer of separated particles separated by a centrifuge apheretic system according to an embodiment.

FIG. 5 is a flow process of an example method for implementing a centrifugal apheretic system according to an embodiment.

FIGS. 6A-6B illustrate an example hydro-cyclone centrifuge design according to an embodiment.

FIG. 7 illustrates an example apheretic biopsy system incorporating multiple hydro-cyclone devices according to an embodiment.

FIGS. 8A-8B illustrate example designs of a hydro-cyclone devices according to an embodiment.

FIG. 9 illustrates an example hydro-cyclone based system according to an embodiment.

FIGS. 10A-B illustrates views of an example collection and waste container.

FIG. 11 is an example method for implementing a hydro-cyclone centrifuge system according to an embodiment.

FIG. 12 is a block diagram of a special-purpose computer system according to an embodiment according to an embodiment.

### DETAILED DESCRIPTION

A centrifuge generally includes a device that uses centrifugal force to separate components or particles in a fluid. For example, a centrifuge-based system can be used to separate blood components in a biological fluid containing cells, such as red blood cells, nucleated cells, and plasma. Centrifugation techniques can either separate plasma from cells, platelets, or cellular components. Regardless of application, these technologies can use a large bowl or a similar object to isolate particles by centrifugation.

The present embodiments relate to various centrifugal-based separation techniques used as part of an apheretic biopsy system or an apheretic therapeutic system to isolate target cell populations. For example, a centrifugal apheretic system as described herein can obtain a biological fluid containing cells from a patient, separate particles in the biological fluid containing cells by one or more particle parameters (e.g., a size, density) and/or other cellular properties using a centrifugal device (or series of connected centrifuge devices) and return a portion of the biological fluid containing cells to the patient while also collecting a relevant portion of the blood (e.g., a buffy coat layer). In some instances, the system as described herein can be directed to a patient, where blood is input directly from the patient, and a remaining portion of the blood is returned to the patient via a return line.

The present embodiments can also be applicable to isolating other blood components with slight modifications to the systems and method as described herein. For instance, the present embodiments can introduce specialized centrifugation methods to enable miniaturization of the centrifuge process and integrate the centrifuge process into the apheretic cell enrichment system. The apheretic system can be designed to enrich and isolate certain types of cells (e.g., nucleated cells) from the liquid that carries the cells (e.g., blood) based on their different physical properties (e.g., density, size), in comparison to the main content of the liquid (e.g., plasma, red blood cells). In the present embodiments, multiple centrifuge design types can be implemented for an apheretic cell enrichment system including, for example, (1) a robotic-assisted centrifuge design, (2) an active spinning centrifuge design, and (3) a hydro-cyclone design. Different designs of centrifuge as described herein can be coupled or integrated with various microfluidic-based apheretic systems to achieve desired selectivity of cells with desired throughput.

Other embodiments could apply to therapeutic applications, for instance, the system could be utilized to filter any tumor cells shed during cancer surgeries or could be utilized to remove excessive leukemia cells from a patient's blood stream. The system could also be applied to filter unwanted cells in other bodily fluids including the cerebrospinal fluid.

### Robotic-Assisted Centrifuge

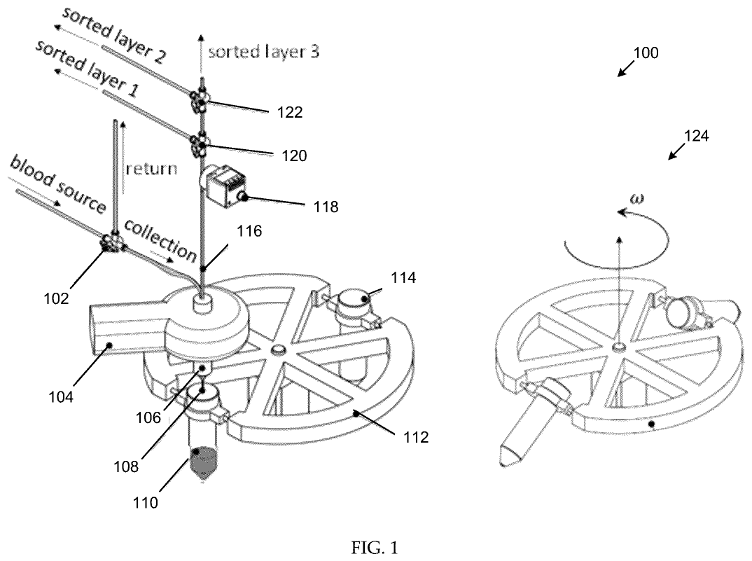

As noted above, a first example centrifuge design can include a robotic-assisted centrifuge design. FIG. 1 illustrates a view 100 of a robotic-assisted centrifuge design. For instance, the design 100 in FIG. 1 can include a blood source being directed into a motorized three-way valve 102. The valve 102 can obtain a blood source and direct the blood into either a return (e.g., return via valve 102) or a collection (e.g., 110).

After input at the blood source, the collected blood can be directed into a robotic arm 104, where the blood is further directed from the robotic arm 104 into the infuse/withdraw system 106. The infuse/withdraw system 106 can be connected to the dual needle 108 and a collecting container 110. Further, the system 100A can include a centrifuge wheel 112 and a second collecting container 114.

From the robotic arm 104, the system can include a withdraw catheter 116, an imaging system 118, and a motorized three-way valve 120 connected to the catheter 116. The valve 120 can direct a fluid to a first sorted layer or a second three-way valve 122.

Further, as shown in FIG. 1, a second centrifuge system 124 can revolve when the other components of the system are loading. The second centrifuge system 124 can rotate about a vertical axis in a direction ω.

As illustrated in FIGS. 1A-1B, the design can include two identical spinning centrifuge wheels (e.g., 112, 124) arranged in a tandem fashion. A first wheel 112 can be connected to a robotic blood injection system, with the second wheel 124 being part of a transport device responsible for injecting unprocessed blood and transporting centrifuged blood samples. Each centrifuge wheel can hold multiple collecting containers which can be used to collect blood samples from the patient.

During an operation, the dual needle (e.g., 108) comprising a first infusing needle and a second extracting needle mounted on the robotic device can be first aligned with the collecting container (e.g., 110) and is inserted into the container. The dual needle 108 can include two needles, which can be used for infusing blood and extracting different layers of blood separately. The motorized three-way valve (e.g., 102) can open, and blood can flow to the collecting container 110 through the infusing needle (e.g., a first needle that is part of the dual needle 108). In some instances, while a dual needle is described, a single needle or multiple separate needles can be incorporated in the present system.

When blood infusion is complete, the needle 108 can be removed from the container by the robotic arm 106 and the centrifuge wheel 112 that holds the containers can start spinning to separate blood components into three layers (e.g., red blood cells (RBCs), buffy coat, and plasma), based on their inertia. After the centrifuging step is finished, the dual needle 108 can be again aligned with the blood sample container 110, the extracting needle can be inserted to the bottom of the container to start the blood extracting process. During this process, an imaging camera (e.g., 118) can be used to monitor the separated layers of blood going through the blood extracting line based on their visual properties (i.e., clarity and darkness). That is each specific layer can have different clarity and/or darkness that can be detected to differentiate the layers. The signal from the camera 118 can be employed to control the three-way valve (e.g., 120) to direct the flow of separated RBCs (sorted layer 1) and plasma (sorted layer 3) back to the patient while the buffy coat (sorted layer 2) can be collected.

As shown in FIG. 1, an imaging system 118 can control the operation of a valve 120 based on the particles flowing through the output 116. The imaging system 118 can include an image sensor (e.g., a camera) and a corresponding computing node. The computing node can be local to the imaging device 118 electrically connected remotely to imaging device (e.g., part of a cloud computing system).

The imaging system 118 can capture one or more images (or video) of the blood cells flowing through output 116 and pass the image(s) to the computing node. The computing node can process the obtained images to determine a layer of particles flowing through the outlet. For example, the computing node can determine whether the blood cells comprise a buffy coat layer or another layer based on a particle size of the depicted particles. Further, the computing node can cause the valve(s) 120, 122 to move based on the determination of the layer of blood particles in the outlet 116. For example, the motor can open valve 120 to direct a first layer of blood cells to a first output. Similarly, a motor can open valve 122 to direct another layer of blood cells to a second output or a third output.

To keep a streamlined operation of the apheretic system, the two centrifuge wheels 112, 124 can alternate between the centrifuging mode and blood infusing/extracting mode. For instance, when one centrifuge wheel 112 operates in the centrifuging mode, the other wheel 124 can operate in the blood infusing/extracting mode. In some instances, each centrifuge wheel can host multiple (e.g., 2, 3, 6, 8, 10, 12, or more) collecting containers (e.g., 110, 114). The number of collecting containers can be adjusted to allow the time that the system is in infusing/extracting mode matches that for centrifuging, which ensures a continuous operation of the apheretic system.

In the robotic-assisted design, the system can include any of: (1) robotic-assisted control of blood infusion and extraction process; (2) imaging-based control of buffy coat separation from RBC layer and plasma layer after the centrifuge step; and (3) a tandem design to ensure continuous operation of the apheretic system.

### Active Spinning Centrifuge

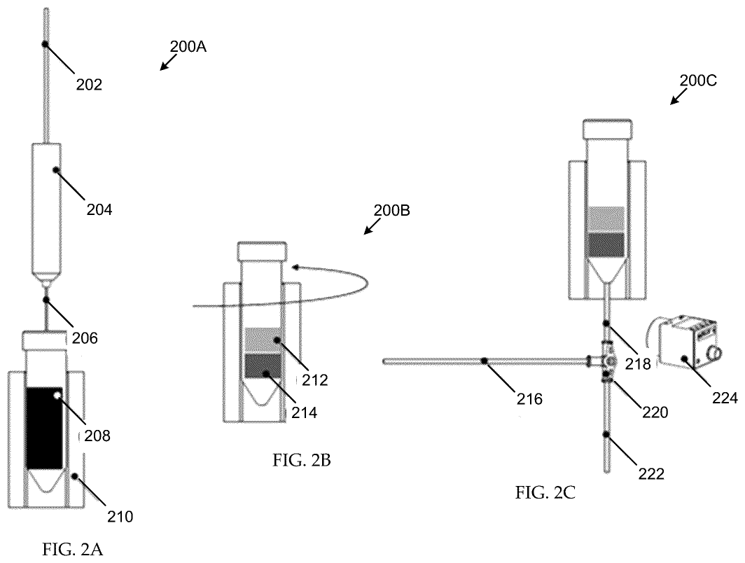

Another example design can include an active spinning centrifuge design. FIGS. 2A-2C illustrate example views 200A-C of an active spinning centrifuge design. Each design as shown in the views 200A-C can comprise two or more identical active spinning sample collecting containers in a tandem fashion.

For example, a first design as shown in FIGS. 2A-2C can depict a center-axis spinning centrifuge. As shown in FIG. 2A, the design 200A can include an infuse catheter 202, and a needle to tube catheter 204 connected to the infuse catheter 202. The design 200A can also include an injecting needle 206 connected to the catheter 204, and a collecting container 208 disposed within a spinner 210.

A center-axis spinning centrifuge 200B can include a collecting container being capable of rotating about a central axis. For instance, separated layers of blood 212, 214 can be separated due to the rotation of the centrifuge. Further, as shown in FIG. 2C, an outlet catheter 218 can output from the container (e.g., 208). A three-way motorized valve 220 can be connected to the outlet catheter 218, an exit catheter to waste 216, and an exit catheter to collection 222. An imaging system 224 can be connected to the three-way motorized valve 220.

For the center-axis design shown in FIGS. 2A-2C, each collecting container (e.g., 208) can include two openings, connecting to the blood infusing (e.g., 206) and blood extracting lines (218), respectively. The collection containers can include passive one-way valves to permit leak-free insertion and retrieval of fluids. Patient blood can be first infused into the container from the top infusion line. The container can spin on its central axis (e.g., as shown in 200B) to suspend the cells, and then the cells can settle by gravity to, in some aspects, three layers (e.g., RBC layer (bottom), buffy coat (middle), and plasma layer (top)). Different layers of blood in the processed sample can be extracted from the extraction line connecting to the bottom of the container. While the connection can be made at the bottom of the container as is shown in FIGS. 2A-2C, in some instances, the connection can be made from the top of the container.

An imaging setup 224 can be used to monitor the blood going through the blood extracting line based on their visual properties (e.g., clarity and darkness) and divide them into the return line (RBC layer and plasma layer) and the collection line (buffy coat). The collecting containers can alternate between the centrifuging mode and blood infusing/extracting mode. In other words, when one container operates in the centrifuging mode, the other one operates in the blood infusing/extracting mode.

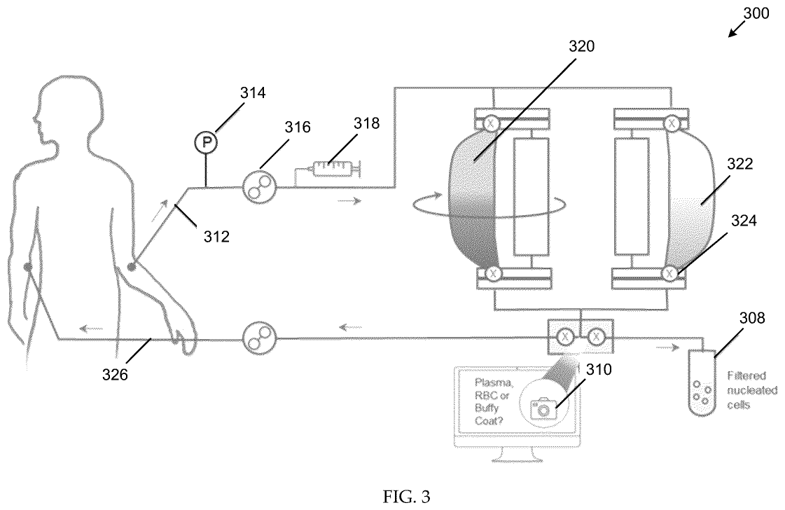

FIG. 3 illustrates an example off-center active spinning centrifuge 300. As shown in FIG. 3, the design 300 can include a collecting container 308 and an imaging system 310 to control a motorized valve. Further, the design can include an apheretic outlet line 312, a pressure sensor 314, a pump 316, and an anti-coagulant injector 318. The design can further include multiple off-center spinning chambers 320, 322, and valves 324 returning at least a portion of a fluid to an apheretic return line 326.

For the off-center design shown in FIG. 3, the spinning containers 320, 322 can be spun-off-center. Blood can be pumped out of the body via pumps (e.g., 316) and then fed to tandem spinning containers 320, 322. Similar to the center axis design, the system can include blood infusing and extracting lines located above and below the spinning containers, and an imaging setup 310 used to monitor the blood going through the extracting lines. Pressure sensors (e.g., 314) can monitor the flow of blood in and out of the patient. Anti-coagulants (e.g., from 318) can be added to ensure blood does not coagulate as it is separated.

This design can include any of: (1) an imaging-based control of buffy coat separation from RBC layer and plasma layer after the centrifuge; and (2) the tandem multi-centrifuge design to ensure the continuous operation of the apheretic system.

As described above, a centrifugal apheretic system is provided. The centrifugal apheretic system can include an inlet (e.g., blood source, 312) configured to obtain a biological fluid containing cells (e.g., from a patient). The system can also include a collection container (e.g., 110) configured to collect the biological fluid containing cells from the inlet.

The system can further include a centrifuge device (e.g., 112, 210, 320, 322) connected to the collection container. The centrifuge device can be configured to rotate about an axis (e.g., a central axis, an off-center axis for the devices 320, 322 in FIG. 3), causing particles in the biological fluid containing cells to separate in the collection container by a particle size and/or a particle flow rate.

In some instances, the centrifuge device is a center axis centrifuge device (e.g., 112) configured to rotate about a central axis. In such instances, the collection container (e.g., 110) is disposed at a periphery of the center axis centrifuge device 112. In some instances, the centrifuge device is a spinning centrifuge device (e.g., 210) configured to surround the collection container 208 and rotate the collection container about the central axis. In some instances, the centrifuge device is an off-center axis centrifuge device (e.g., 320, 322) configured to rotate offset relative to the central axis. In some instances, the system can include at least two off-center axis centrifuge devices (e.g., 320, 322) connected to the inlet in parallel. Each of the at least two off-center axis centrifuge devices can include collection containers, and each collection container can be configured to receive part of the biological fluid containing cells.

The system can further include an outlet (e.g., 116) configured to direct a first portion of the separated particles in the biological fluid containing cells from the collection container to a first channel of the outlet and direct a second portion of the separated particles in the biological fluid containing cells from the collection container to a second channel of the outlet. A valve (e.g., 120) can control the flow of the particles from a first channel or a second channel based on a determined particle size.

In some instances, the system can include a robotic arm (e.g., 104) connected to the inlet and a dual needle (e.g., 108) connected to the robotic arm. The robotic arm 104 can be configured to move the dual needle 108 into the collection container 110. Further, a first needle of the dual needle can be configured to provide the biological fluid containing cells to the collection container, and a second needle of the dual needle can be configured to direct the separated particles from the collection container to the outlet.

In some instances, the system can include a motorized valve (e.g., 120, 122) connected to the outlet. The motorized valve can be configured to open to either the first channel to direct the first portion of the separated particles to the first channel or the second channel to direct the second portion of the separated particles to the second channel. The first portion of the separated particles can include a buffy coat layer of particles of the biological fluid containing cells.

In some instances, the system can include an image sensor (e.g., 118) connected to the motorized valve (e.g., 120, 122) and configured to capture an image of the separated particles of the biological fluid containing cells. The system can also include a computing node electrically connected to the sensor. The computing node can include a processor and a memory. The memory can include instructions that are configured to cause the processor to perform a series of steps.

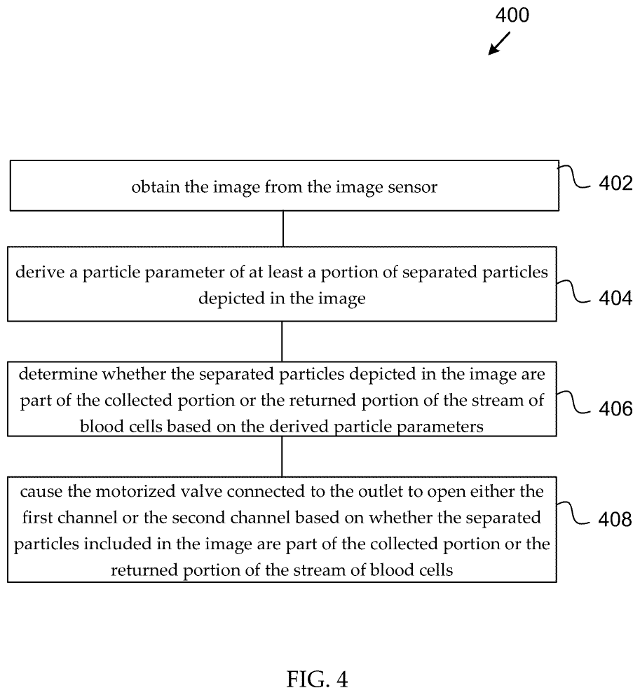

FIG. 4 is an example flow process 400 for moving a motorized valve based on determined properties of the particles separated by a centrifuge apheretic system. The imaging system can capture visual differences between the flow (or particles included) of the different layers separated by the systems as described herein. The visual differences can include a difference of the flow and the separated particles in the flow.

At 402, the method can include obtaining the image from the image sensor. The image sensor can capture an image, series of images, and/or a video depicting one or more particles in the outlet. In some instances, the imaging system can use infrared (IR) light sources and one or more cameras to capture changes in hematocrit levels in the fluid to detect separated layers of particles.

At 404, the method can include deriving a parameter of at least a portion of separated particles depicted in the image. The computing node can perform imaging processing to determine a flow of the particles depicted, an average size of particles identified in the input image, etc. The flow of the layer of particles can specify a layer of blood cells separated by the centrifuge and provided at the outlet.

At 406, the method can include determining whether the separated particles depicted in the image are part of the a collected portion or the returned portion of the biological fluid containing cells based on the derived particle parameters. The collected portion of the biological fluid containing cells can include cells for further testing to be collected in a collection container. The returned portion of the biological fluid containing cells can include blood cells that are not to be collected (e.g., not part of the buffy coat layer) and can be returned to the patient. In some instances, another portion of the biological fluid containing cells can include another layer of cells separated by the one or more centrifuge devices. The computing node can determine whether the particle parameter specify cells that are part of a layer of cells to be collected or returned.

At 408, the method can include the motorized valve connected to the outlet to open either the first channel or the second channel based on whether the separated particles included in the image are part of the collected portion or the returned portion of the biological fluid containing cells. For example, if the cells are part of the collected portion of blood cells (e.g., the buffy coat layer), the valve can be opened to a first position to outlet to a first channel. If the cells are part of the returned portion of blood cells (e.g., not including the buffy coat layer), the valve can be opened to a second position to outlet to a second channel.

In some instances, the first channel can be configured to direct the collected portion of the separated particles to another container for further testing, and the second channel is configured to direct the returned portion of the separated particles to a patient via a return line.

In another example embodiment, a system is provided. The system can include an inlet configured to obtain a biological fluid containing cells. The system can also include a collection container configured to collect the biological fluid containing cells from the inlet. The system can also include a centrifuge device connected to the collection container. The centrifuge device can be configured to rotate about an axis, causing particles in the biological fluid containing cells to separate in the collection container.

The system can also include an outlet comprising a first channel and a second channel. The system can also include a motorized valve configured to open the first channel and the second channel. The system can also include an image sensor connected to the motorized valve and configured to capture an image of the separated particles of the biological fluid containing cells.

The system can also include a computing node electrically connected to the sensor, wherein the computing node comprises a processor and a memory. The memory can include instructions that are configured to cause the processor to obtain the image from the image sensor, derive a particle size of at least a portion of separated particles depicted in the image, determine whether the separated particles depicted in the image are part of the first portion or the second portion of the biological fluid containing cells based on the derived particle sizes, and cause the motorized valve connected to the outlet to open either the first channel or the second channel based on whether the separated particles included in the image are part of the first portion or the second portion of the biological fluid containing cells.

In some instances, the outlet can be configured to direct a first portion of the separated particles in the biological fluid containing cells from the collection container to the first channel of the outlet and direct a second portion of the separated particles in the biological fluid containing cells from the collection container to the second channel of the outlet.

In some instances, the system can include a robotic arm connected to the inlet; and a dual needle connected to the robotic arm. The robotic arm can be configured to move the dual needle into the collection container. A first needle of the dual needle can be configured to provide the biological fluid containing cells to the collection container, and a second needle of the dual needle is configured to direct the separated particles from the collection container to the outlet.

In some instances, the centrifuge device is a center axis centrifuge device configured to rotate about a central axis, and wherein the collection container is disposed at a periphery of the center axis centrifuge device. In some instances, the centrifuge device is a spinning centrifuge device configured to surround the collection container and rotate the collection container about the central axis.

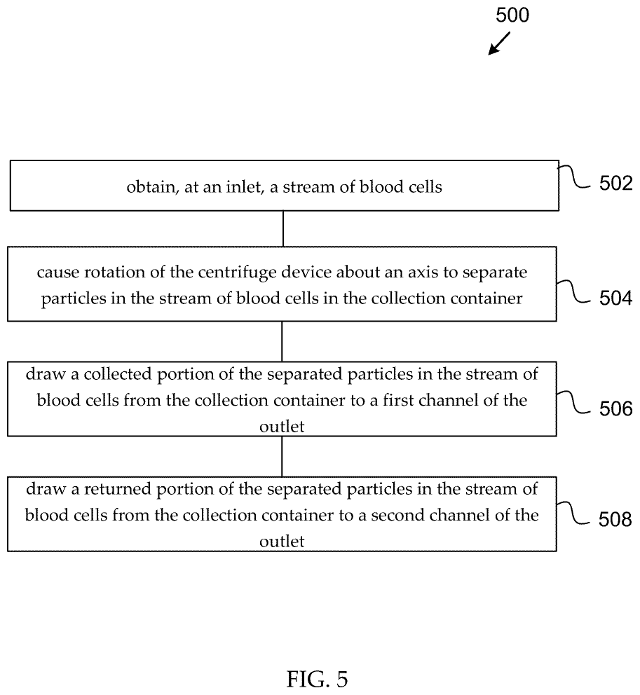

FIG. 5 is a flow process 500 of an example method for implementing a centrifugal apheretic system. At 502, the method can include obtaining, at an inlet, a biological fluid containing cells. The biological fluid containing cells can be directed from the inlet to a collection container connected to a centrifuge device.

At 504, the method can include causing rotation of the centrifuge device about an axis to separate particles in the biological fluid containing cells in the collection container. In some instances, the method can also include pressurizing, by a pump, the biological fluid containing cells in the inlet. The biological fluid containing cells can be obtained at the inlet from a patient. The second portion of the separated particles in the biological fluid containing cells can be directed from the second channel to the patient.

In some instances, the method can also include moving, by a robotic arm connected to the inlet and to a dual needle, the dual needle into the collection container. A first needle of the dual needle can be configured to provide the biological fluid containing cells to the collection container and a second needle of the dual needle can be configured to direct the separated particles from the collection container to the outlet. In some instances, the method can also include disposing, by an injector, an anti-coagulant into the biological fluid containing cells in the inlet.

At 506, the method can include drawing a collected portion of the separated particles in the biological fluid containing cells from the collection container to a first channel of the outlet.

At 508, the method can include drawing a returned portion of the separated particles in the biological fluid containing cells from the collection container to a second channel of the outlet. In some instances, the method can also include opening, by a motorized valve connected to the outlet, either the first channel to direct the first portion of the separated particles to the first channel or the second channel to direct the second portion of the separated particles to the second channel. The first portion of the separated particles can include a buffy coat layer of particles of the biological fluid containing cells.

In some instances, the method can include capturing, by an image sensor connected to the outlet, an image of the separated particles of the biological fluid containing cells. The method can also include obtaining, by a computing node, the image from the image sensor. The method can also include deriving, by the computing node, a particle size of at least a portion of separated particles depicted in the image. The method can also include determining, by the computing node, whether the separated particles depicted in the image are part of the first portion or the second portion of the biological fluid containing cells based on the derived particle sizes. The method can also include causing, by the computing node, the motorized valve connected to the outlet to open either the first channel or the second channel based on whether the separated particles included in the image are part of the first portion or the second portion of the biological fluid containing cells.

### Hydro-Cyclone Centrifuge

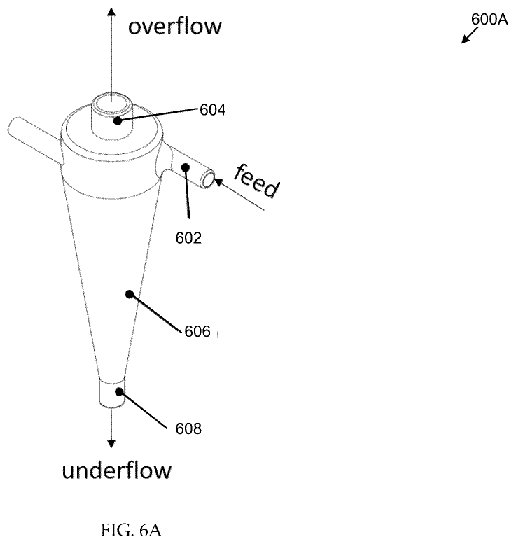

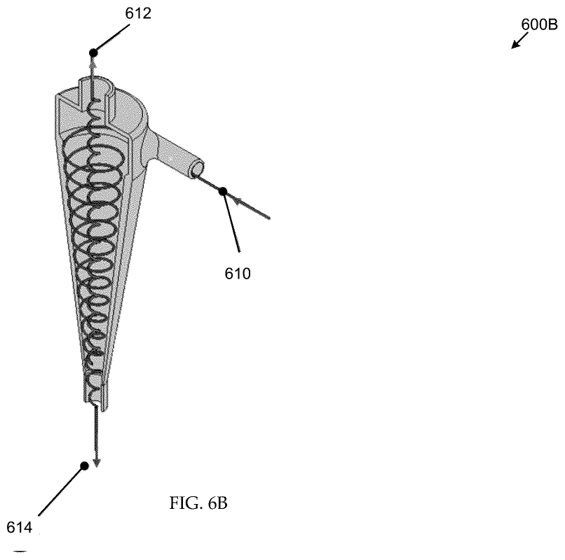

As described above, an example design can include a hydro-cyclone centrifuge design. FIGS. 6A-6B illustrate an example hydro-cyclone centrifuge design. As shown in FIG. 6A, the centrifuge 600A can include a feed 602, an overflow outlet 604, a body portion 606, and a nozzle 608. Further, in the cross-section view in FIG. 6B, the centrifuge 600B can include a blood stream 610, a vortex of the lower mass particles/cells 612, and a vortex of larger particles/cells 614.

As illustrated in FIGS. 6A-6B, the hydro-cyclone can include a conical section (e.g., 606) connected to a cylindrical section. The cylindrical section can be connected to one or two tangential inlet(s) (e.g., 602) and an overflow orifice (e.g., 604). The conical section can end with an underflow orifice (e.g., 608). The entire hydro-cyclone may not include any moving parts. Rather, it can use the centrifugal force generated during the fluid moving tangentially into the conical chamber to separate particles with different density and size. The hydro-cyclone as described herein can be implemented for blood cell separation (i.e., separation of WBCs from the remaining blood component), and can be integrated into an apheretic system.

A design of the hydro-cyclone can be used to achieve effective blood cell separation and integrate it into an apheretic system. For instance, the present design can have the blood sample fed into the hydro-cyclone through the tangential inlets. The angular momentum carried by the sample can generate a primary vortex (e.g., as illustrated in FIG. 6B), which can push heavier and larger size particles in the blood sample outward and downward (e.g., 614) alongside the wall of the conical section and eventually exiting from the underflow orifice of the hydro-cyclone. In the meantime, a secondary vortex can be formed in the center of the hydro-cyclone which moves the lighter and smaller particles upward (e.g., in 612) and pushes them out through the overflow orifice. Since the particle separation in hydro-cyclone can be more size dependent than density dependent, the large size cells in the blood (e.g., WBCs and circulating tumor cells) can be collected in the underflow (e.g., 608) while the small cells and lighter fluid (e.g., RBCs and plasma) can be collected in the overflow (e.g., 604).

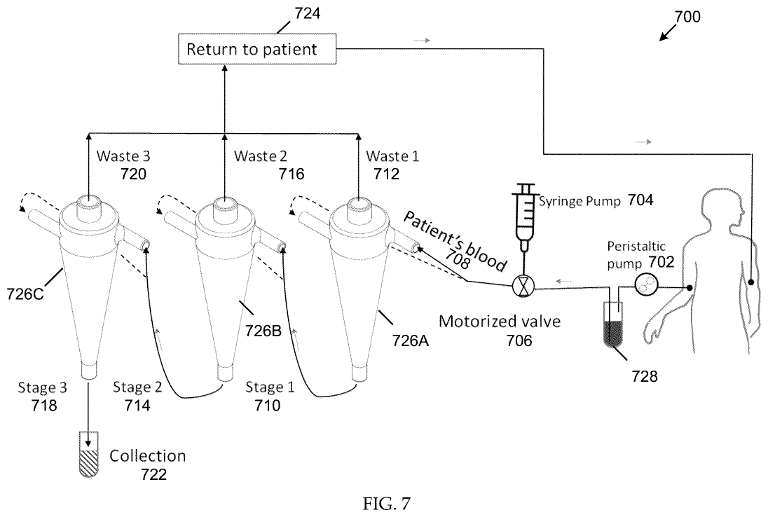

FIG. 7 illustrates an example apheretic biopsy system incorporating multiple hydro-cyclone devices. For example, three hydro-cyclone devices can be connected in series to separate particles in a blood stream in multiple stages. While three devices are shown, any number of devices can be incorporated into the present system.

As shown in FIG. 7, blood can be drawn from the patient with through a pump 702 and collected in a buffer container 728. A motorized valve 706 can rotate and connect the container to the apheresis system 700. For instance, the patient blood 708 can be directed into a first hydro-cyclone device 726A, with pressure provided by the pump 702 (e.g., peristaltic pump). Further, a syringe pump 704 can provide an anti-coagulant and/or provide pressure to the blood 708 via valve 706.

At the first hydro-cyclone device 726A, a first portion of cells (e.g., cells part of the buffy coat layer) can be output at a first stage 710, while a remaining portion of the cells can be outputted at waste 712. The first stage 710 can input into a second hydro-cyclone device 726B, which can further filter another portion of cells (e.g., cells part of the buffy coat layer) to be output at a second stage 714, while a remaining portion of the cells can be outputted at waste 716. The second stage 716 can input into a third hydro-cyclone device 726C, which can further filter another portion of cells (e.g., cells part of the buffy coat layer) to be output at a third stage 718 and into a collection container 722, while a remaining portion of the cells can be outputted at waste 720. The remaining portion of the cells (e.g., cells output at wastes 712, 716, 720) can be returned to patient (e.g., at 724).

To improve the efficacy of the hydro-cyclone for blood cell separation, various changes to the design of each hydro-cycle can be made. For instance, the device size can be reduced, which can allow for separation of the cells with smaller size and density difference under a lower flow rate. Further, a conical angle and conical section length can be reduced, which can influence the flow rates of underflow and overflows and separation efficiency. Additionally, a flow rate can be optimized in the device(s) to allow for seamless integration with the apheretic system.

The hydro-cyclone system can also be designed to operate in a serial and/or parallel fashion to improve the separation efficiency or the throughput of separated particles. Specifically, for a serial configuration as illustrated in FIG. 7, the underflow collected in a hydro-cyclone can be fed into the sample inlet of a second hydro-cyclone to further concentrate the larger and denser particles in the collection. In an apheretic system, the number of hydro-cyclones (e.g., 2, 3, 4, 5, 6, 7, 8 or more) can be selected to allow the total sample processing flow rate to match specifications for a therapeutic apheresis process. In addition, as shown in FIG. 7, to cope with the potential mismatch between the optimal flow rate for the hydro-cyclone and the flow rate for operating the apheretic system, a buffer container (e.g., 728) can be added between the patient blood extraction line and the syringe pump that drives the fluid motion in the hydro-cyclones.

The system incorporating one or more hydro-cyclones can incorporate one or more hydro-cyclone devices for blood cell separation. Further, a design of the hydro-cycles can be modified (e.g., modifying a diameter, conical angle, and conical section length, and flow rate) to achieve a desired efficiency for cell isolation. The cylindrical chamber can be defined by two variables that determine the diameter and the length of the chamber. The length is in the range of 1 mm to 50 mm, and the diameter is in the range of 1 mm to 50 mm. The length of the conical chamber that is integrated with the cylindrical chamber can be in the range of 5 mm to 300 mm. There can include two inputs that merge into the cylindrical chamber. Their inner diameter is in the range of 0.3 mm to 3 mm. There can also include two outputs; one connected to the cylindrical chamber, and one is connected to the conical chamber. The inner diameter of the outputs is in the range of 0.3 mm to 3 mm. Additionally, the system can integrate the hydro-cyclone in an apheretic system for continuous blood processing. In some instances, the system can use hydro-cyclones with different optimized parameters in series in one device.

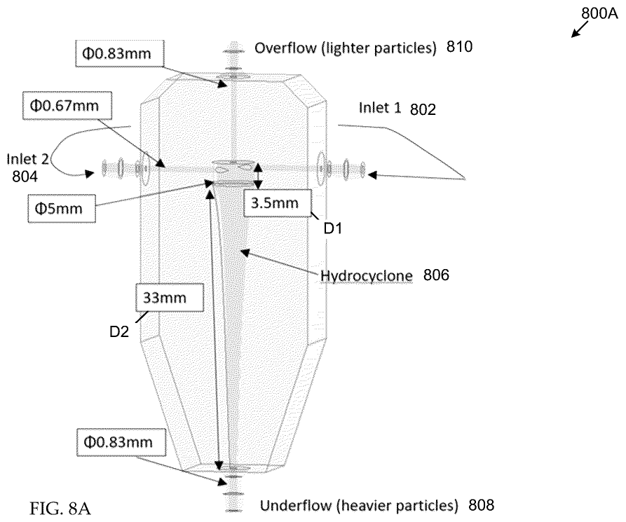

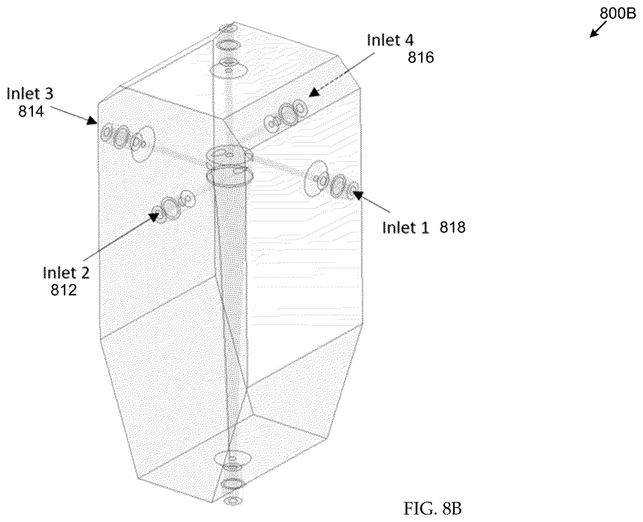

As described above, a hydro-cyclone device can incorporate various parameters. FIGS. 8A-8B illustrate example designs of a hydro-cyclone devices 800A-B. For example, in FIG. 8A, a first design 800A can include a hydro-cyclone device with two inlets 802, 804. The hydro-cyclone component 806 can obtain blood from inlets 802, 804, with heavier particles emitting from an underflow 808 (e.g., buffy coat layer particles) and lighter particles emitting from an overflow 810 (e.g., remaining blood cells).

In some instances, as shown in FIG. 8A, a height of a cylindrical portion D1 of the hydro-cyclone 806 can be around 3.5 mm, a diameter of the inlets can be around 0.67 mm, and a diameter of an overflow and/or an underflow can be around 0.83 mm. A diameter of the cylindrical portion of the hydro-cyclone 806 can be around 5 mm, and a length of the conical portion of the hydro-cyclone 806 can be around 33 mm.

FIG. 8B illustrates a second example hydro-cyclone device design 800B. As shown in FIG. 8B, the device 800B can include four inlets 812, 814, 816, 818. The multiple inlets can increase vortex speed in the hydro-cyclone. Further, spiral channels can be added inside the hydro-cyclone to increase its particle separation capability.

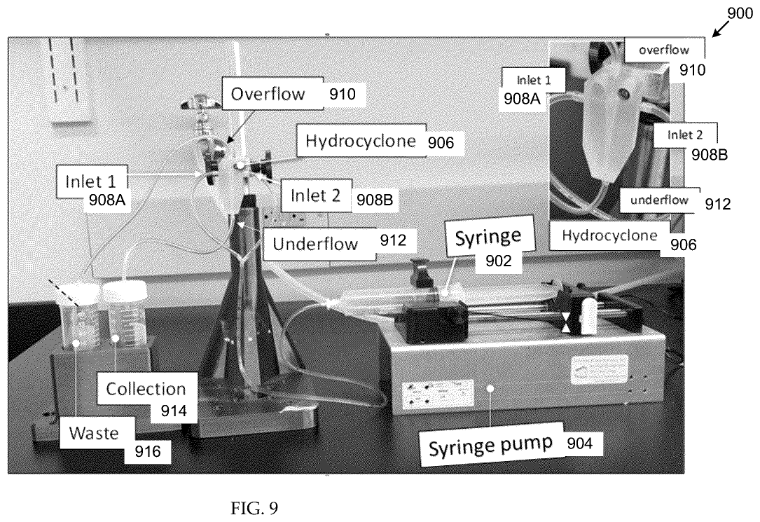

FIG. 9 illustrates an example hydro-cyclone based system. As shown in FIG. 9, the system 900 can include a syringe 902, syringe pump 904, and a hydro-cyclone 906. The hydro-cyclone 906 can include inlets 908A-B, an overflow 910, and an underflow 912. The system 900 can also include a collection 914 and a waste 916.

As an example, the system as described herein can incorporate miniature hydro-cyclone devices that is capable of separating particles (e.g., beads) with differing sizes (e.g., 3 um beads and 8 um beads). The syringe pump can operate at 1 mL/s, or at a range between 1-2 mL/s (e.g., about 1.0, 1.1., 1.2, 1.3, 1.4., 1.5, 1.6, 1.7, 1.8, 1.9, 2.0 mL/s or more), for example.

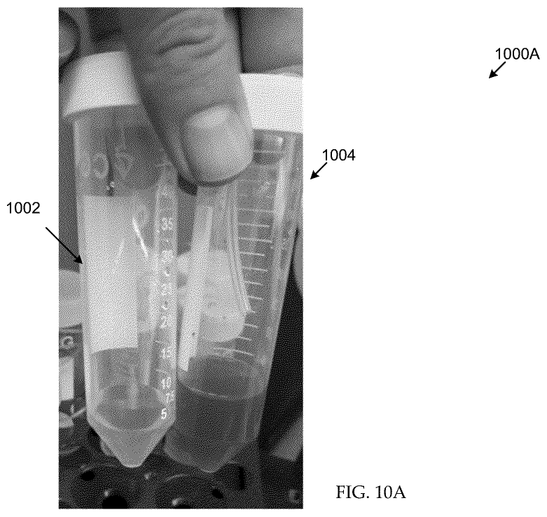

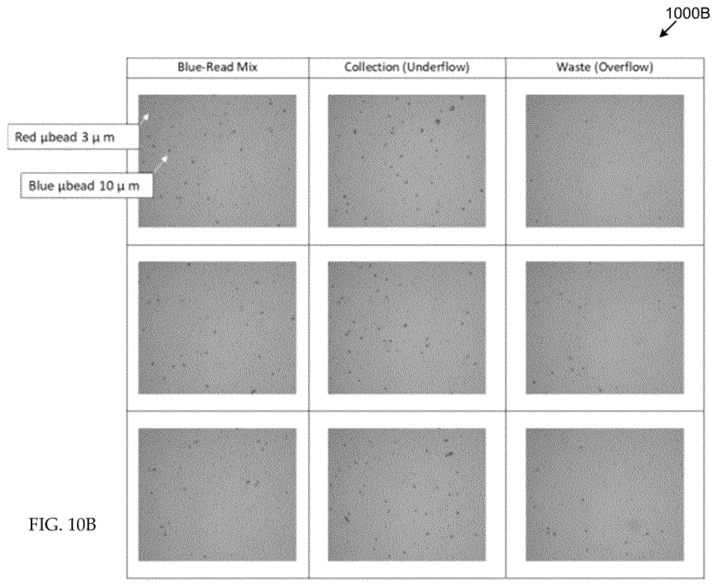

FIG. 10A illustrates a view 1000A of an example collection and waste container. As shown in FIG. 10A, a first portion of a material can be fed into a collection 1004, while a second portion of the material can be fed into a waste 1002. The material can be separated by particle size and/or flow rate. Further, FIG. 10B illustrates an example depiction of samples of a blue-red mix, collection, and waste.

In some instances, material collected from the underflow can be filled with more concentrated blue particles than that from the overflow. With increasing flow rate (which can lead to increasing separation capability), the difference in concentration between the underflow and overflow can also increase. In another embodiment, a mixture of 3 μm red particles and 8 μm blue particles can be fed into the inlets of the hydro-cyclone at 1 mL/s. The results can be compared to the images from the original sample. Further, the sample collected from the overflow can contain mostly red particles while that collected from the underflow contains mostly larger blue particles.

In some instances, a hydro-cyclone apheretic system is provided. The hydro-cyclone apheretic system can include an inlet configured to obtain a biological fluid containing cells from a patient. The system can also include a first hydro-cyclone centrifuge device (e.g., 726A) connected to the inlet.

The first hydro-cyclone centrifuge device can include at least one intake (e.g., 602) to obtain at least a portion of the biological fluid containing cells at a cylindrical portion of the first hydro-cyclone centrifuge device. The first hydro-cyclone centrifuge device can also include an overflow (e.g., 604) disposed at a top portion of the cylindrical portion. A first portion of the biological fluid containing cells can be configured to be separated in the first hydro-cyclone centrifuge device and output at the overflow. The first hydro-cyclone centrifuge device can also include an underflow (e.g., 608) disposed at a bottom portion of a conical portion of the first hydro-cyclone centrifuge device. A second portion of the biological fluid containing cells can be configured to be separated in the first hydro-cyclone centrifuge device and output at the underflow.

The system can also include an outlet (e.g., 724) connected to the overflow. The first portion of the biological fluid containing cells can be configured to be output from the overflow to the outlet and returned to the patient via a return line. The system can also include a first collection container (e.g., 722) connected to the underflow. The second portion of the biological fluid containing cells can be configured to be output from the underflow to the first collection container.

In some instances, the first hydro-cyclone centrifuge device comprises two intakes, with each intake being disposed at an opposing end of the cylindrical portion.

In some instances, the system can also include a pump (e.g., 702) configured to pressurize the biological fluid containing cells in the inlet to a specified flow rate range of between 1-2 milliliters per second (mL/s).

In some instances, the system can also include a second collection container (e.g., 728) configured to collect the biological fluid containing cells from the patient. The biological fluid containing cells can be configured to be directed to the inlet from the second collection container.

In some instances, the cylindrical portion comprises a height of between 3-5 mm (e.g., 3.0, 3.5, 4.0, 4.5, 5.0 mm or more), and wherein the conical portion comprises a height between 30-35 mm (e.g., about 30, 31, 32, 33, 34, or 35 mm).

In some instances, the system can also include a second hydro-cyclone centrifuge device (e.g., 726B). An intake of the second hydro-cyclone centrifuge device can be connected to the underflow of the first hydro-cyclone centrifuge device. The system can also include a third hydro-cyclone centrifuge device (e.g., 726C). An intake of the third hydro-cyclone centrifuge device can be connected to the underflow of the second hydro-cyclone centrifuge device. Further, an overflow of each of the second hydro-cyclone centrifuge device and the third hydro-cyclone centrifuge device can configured to output at the outlet.

In some instances, dimensions of any of the second hydro-cyclone centrifuge device and the third hydro-cyclone centrifuge device differ from dimensions of the first hydro-cyclone centrifuge device.

In some instances, the first hydro-cyclone centrifuge device is configured to cause a vortex of the biological fluid containing cells input at the intake, causing separation of the first portion and the second portion of the biological fluid containing cells based on a particle parameters (e.g., particle size, flow rate, density, and/or deformability).

In another example embodiment, a system is provided. The system can include an inlet configured to obtain a biological fluid containing cells and a series of interconnected hydro-cyclone centrifuge devices.

The series of interconnected hydro-cyclone centrifuge devices can include a first hydro-cyclone centrifuge device connected to the inlet. The first hydro-cyclone centrifuge device can include at least one intake connected to the inlet, an overflow configured to output a first portion of the biological fluid containing cells separated in the first hydro-cyclone centrifuge device, and an underflow configured to output a second portion of the biological fluid containing cells separated in the first hydro-cyclone centrifuge device.

The series of interconnected hydro-cyclone centrifuge devices can also include a second hydro-cyclone centrifuge device with at least one intake connected to the underflow of the first hydro-cyclone centrifuge device. The second hydro-cyclone centrifuge device can be configured to further separate the second portion of the biological fluid containing cells, with a first part being directed to an overflow of the second hydro-cyclone centrifuge device, and a second part being directed to an underflow of the second hydro-cyclone centrifuge device.

The series of interconnected hydro-cyclone centrifuge devices can also include a third hydro-cyclone centrifuge device with at least one intake connected to the underflow of the second hydro-cyclone centrifuge device. The third hydro-cyclone centrifuge device can be configured to further separate the second part of the biological fluid containing cells, with a first part being directed to an overflow of the third hydro-cyclone centrifuge device, and a second part being directed to an underflow of the third hydro-cyclone centrifuge device.

The system can also include an outlet connected to the overflow of each of the series of hydro-cyclone centrifuge devices. The system can also include a first collection container connected to the underflow of the third hydro-cyclone centrifuge device.

In some instances, dimensions of any of the second hydro-cyclone centrifuge device and the third hydro-cyclone centrifuge device differ from dimensions of the first hydro-cyclone centrifuge device.

In some instances, the system can include a pump configured to pressurize the biological fluid containing cells in the inlet to a specified flow rate range of between 1-2 milliliters per second (mL/s) (e.g., about 1.0, 1.1., 1.2, 1.3, 1.4., 1.5, 1.6, 1.7, 1.8, 1.9, 2.0 mL/s or more).

In some instances, the system can include a second collection container configured to collect the biological fluid containing cells from a patient. The biological fluid containing cells can be configured to be directed to the inlet from the second collection container.

In some instances, any of the series of hydro-cyclone centrifuge devices comprise: a cylindrical portion comprising a height of between 3-5 mm (e.g., 3.0, 3.5, 4.0, 4.5, 5.0 mm or more), and a conical portion comprising a height between 30-35 mm (e.g., 30, 31, 32, 33, 34, 35 mm or more).

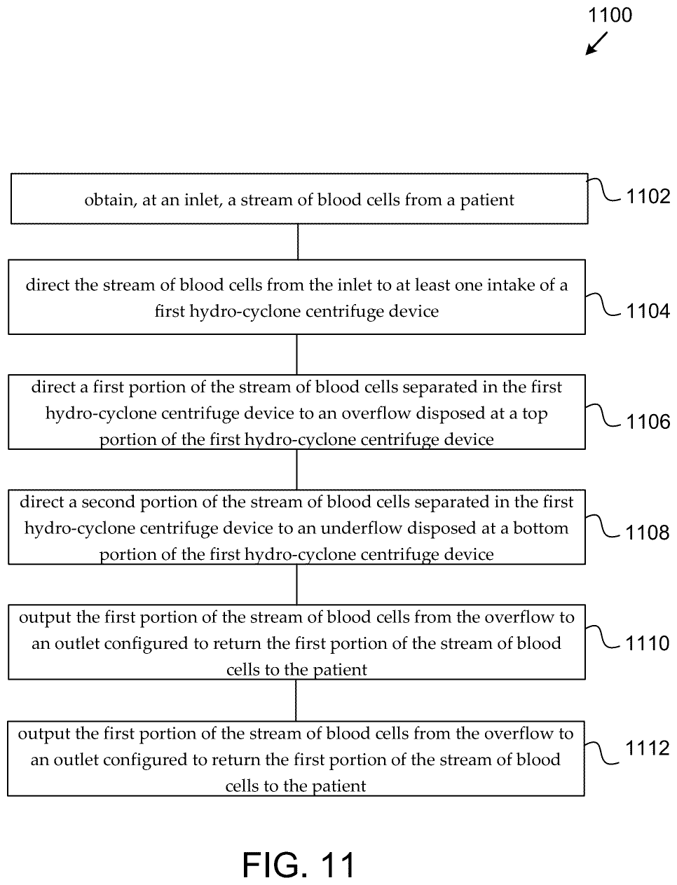

FIG. 11 is an example method 1100 for implementing a hydro-cyclone centrifuge system. At 1102, the method can include obtaining, at an inlet, a biological fluid containing cells from a patient.

In some instances, the method can include pressurizing, by a pump, the biological fluid containing cells in the inlet to a specified flow rate range of between 0.5-5 (or 1-2) milli-liters per second (mL/s).

In some instances, the method can include collecting, at a second collection container, the biological fluid containing cells from the patient. The biological fluid containing cells can be directed to the inlet from the second collection container.

At 1104, the method can include directing the biological fluid containing cells from the inlet to at least one intake of a first hydro-cyclone centrifuge device. The first hydro-cyclone centrifuge device can be configured to cause separation of the biological fluid containing cells.

In some instances, the first hydro-cyclone centrifuge device comprises either two intakes or four intakes, with each intake disposed at opposing ends of the cylindrical portion.

In some instances, the first hydro-cyclone centrifuge device comprises a cylindrical portion and a conical portion. The intake can be connected to the cylindrical portion. The overflow can be connected to a top surface of the cylindrical portion. The underflow can be connected to a bottom surface of the conical portion. In some instances, the cylindrical portion comprises a height of between 3-5 mm, and the conical portion comprises a height between 30-35 mm.

At 1106, the method can include directing a first portion of the biological fluid containing cells separated in the first hydro-cyclone centrifuge device to an overflow disposed at a top portion of the first hydro-cyclone centrifuge device.

At 1108, the method can include directing a second portion of the biological fluid containing cells separated in the first hydro-cyclone centrifuge device to an underflow disposed at a bottom portion of the first hydro-cyclone centrifuge device.

At 1110, the method can include outputting the first portion of the biological fluid containing cells from the overflow to an outlet configured to return the first portion of the biological fluid containing cells to the patient.

At 1112, the method can include outputting the second portion of the biological fluid containing cells from the underflow to a first collection container.

In some instances, the method can include directing the second portion of the biological fluid containing cells from the underflow of the first hydro-cyclone centrifuge device to an intake of a second hydro-cyclone centrifuge device. The second hydro-cyclone centrifuge device can further separate particles in the second portion of the biological fluid containing cells. The method can also include directing a first part of the second portion of the biological fluid containing cells an overflow disposed at a top portion of the second hydro-cyclone centrifuge device. The method can also include directing a second part of the second portion of the biological fluid containing cells to an underflow disposed at a bottom portion of the second hydro-cyclone centrifuge device. The method can also include outputting the first part of the second portion of the biological fluid containing cells from the overflow to the outlet. The method can also include outputting the second part of the second portion of the biological fluid containing cells from the underflow to the first collection container.

### Computing System Overview

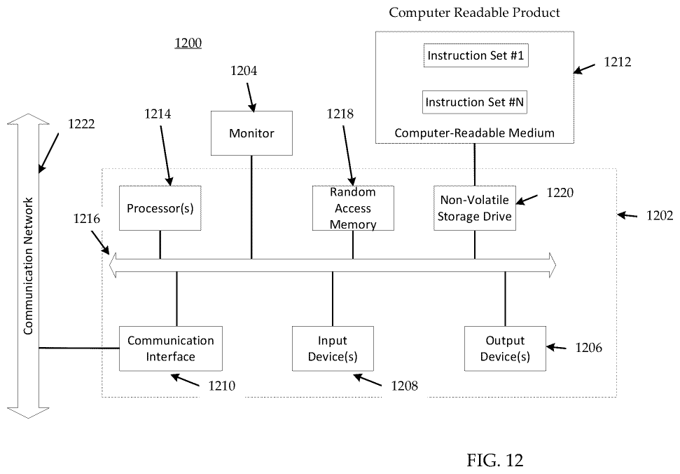

FIG. 12 is a block diagram of a special-purpose computer system 1200 according to an embodiment. For example, system 1200 can deployed as part of a computing node as described herein. The methods and processes described herein may similarly be implemented by tangible, non-transitory computer readable storage mediums and/or computer-program products that direct a computer system to perform the actions of the methods and processes described herein. Each such computer-program product may comprise sets of instructions (e.g., codes) embodied on a computer-readable medium that directs the processor of a computer system to perform corresponding operations. The instructions may be configured to run in sequential order, or in parallel (such as under different processing threads), or in a combination thereof.

Special-purpose computer system 1200 comprises a computer 1202, a monitor 1204 coupled to computer 1202, one or more additional user output devices 1206 (optional) coupled to computer 1202, one or more user input devices 1208 (e.g., keyboard, mouse, track ball, touch screen) coupled to computer 1202, an optional communications interface 1210 coupled to computer 1202, and a computer-program product including a tangible computer-readable storage medium 1212 in or accessible to computer 1202. Instructions stored on computer-readable storage medium 1212 may direct system 1200 to perform the methods and processes described herein. Computer 1202 may include one or more processors 1214 that communicate with a number of peripheral devices via a bus subsystem 1216. These peripheral devices may include user output device(s) 1206, user input device(s) 1208, communications interface 1210, and a storage subsystem, such as random access memory (RAM) 1218 and non-volatile storage drive 1220 (e.g., disk drive, optical drive, solid state drive), which are forms of tangible computer-readable memory.

Computer-readable medium 1212 may be loaded into random access memory 1218, stored in non-volatile storage drive 1220, or otherwise accessible to one or more components of computer 1202. Each processor 1214 may comprise a microprocessor, such as a microprocessor from Intel® or Advanced Micro Devices, Inc.®, or the like. To support computer-readable medium 1212, the computer 1202 runs an operating system that handles the communications between computer-readable medium 1212 and the above-noted components, as well as the communications between the above-noted components in support of the computer-readable medium 1212. Exemplary operating systems include Windows® or the like from Microsoft Corporation, Solaris® from Sun Microsystems, LINUX, UNIX, and the like. In many embodiments and as described herein, the computer-program product may be an apparatus (e.g., a hard drive including case, read/write head, etc., a computer disc including case, a memory card including connector, case, etc.) that includes a computer-readable medium (e.g., a disk, a memory chip, etc.). In other embodiments, a computer-program product may comprise the instruction sets, or code modules, themselves, and be embodied on a computer-readable medium.

User input devices 1208 include all possible types of devices and mechanisms to input information to computer system 1202. These may include a keyboard, a keypad, a mouse, a scanner, a digital drawing pad, a touch screen incorporated into the display, audio input devices such as voice recognition systems, microphones, and other types of input devices. In various embodiments, user input devices 1208 are typically embodied as a computer mouse, a trackball, a track pad, a joystick, wireless remote, a drawing tablet, a voice command system. User input devices 1208 typically allow a user to select objects, icons, text and the like that appear on the monitor 1204 via a command such as a click of a button or the like. User output devices 1206 include all possible types of devices and mechanisms to output information from computer 1202. These may include a display (e.g., monitor 1204), printers, non-visual displays such as audio output devices, etc.

Communications interface 1210 provides an interface to other communication networks and devices and may serve as an interface to receive data from and transmit data to other systems, WANs and/or the Internet, via a wired or wireless communication network 1222. In addition, communications interface 1210 can include an underwater radio for transmitting and receiving data in an underwater network. Embodiments of communications interface 1210 typically include an Ethernet card, a modem (telephone, satellite, cable, ISDN), a (asynchronous) digital subscriber line (DSL) unit, a FireWire® interface, a USB® interface, a wireless network adapter, and the like. For example, communications interface 1210 may be coupled to a computer network, to a FireWire® bus, or the like. In other embodiments, communications interface 1210 may be physically integrated on the motherboard of computer 1202, and/or may be a software program, or the like.

RAM 1218 and non-volatile storage drive 1220 are examples of tangible computer-readable media configured to store data such as computer-program product embodiments of the present invention, including executable computer code, human-readable code, or the like. Other types of tangible computer-readable media include floppy disks, removable hard disks, optical storage media such as CD-ROMs, DVDs, bar codes, semiconductor memories such as flash memories, read-only-memories (ROMs), battery-backed volatile memories, networked storage devices, and the like. RAM 1218 and non-volatile storage drive 1220 may be configured to store the basic programming and data constructs that provide the functionality of various embodiments of the present invention, as described above.

Software instruction sets that provide the functionality of the present invention may be stored in computer-readable medium 1212, RAM 1218, and/or non-volatile storage drive 1220. These instruction sets or code may be executed by the processor(s) 1214. Computer-readable medium 1212, RAM 1218, and/or non-volatile storage drive 1220 may also provide a repository to store data and data structures used in accordance with the present invention. RAM 1218 and non-volatile storage drive 1220 may include a number of memories including a main random access memory (RAM) to store instructions and data during program execution and a read-only memory (ROM) in which fixed instructions are stored. RAM 1218 and non-volatile storage drive 1220 may include a file storage subsystem providing persistent (non-volatile) storage of program and/or data files. RAM 1218 and non-volatile storage drive 1220 may also include removable storage systems, such as removable flash memory.

Bus subsystem 1216 provides a mechanism to allow the various components and subsystems of computer 1202 communicate with each other as intended. Although bus subsystem 1216 is shown schematically as a single bus, alternative embodiments of the bus subsystem may utilize multiple busses or communication paths within the computer 1202.

For a firmware and/or software implementation, the methodologies may be implemented with modules (e.g., procedures, functions, and so on) that perform the functions described herein. Any machine-readable medium tangibly embodying instructions may be used in implementing the methodologies described herein. For example, software codes may be stored in a memory. Memory may be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” may represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

### CONCLUSION

It will be understood that terms such as “top,” “bottom,” “above,” “below,” and x-direction, y-direction, and z-direction as used herein as terms of convenience that denote the spatial relationships of parts relative to each other rather than to any specific spatial or gravitational orientation. Thus, the terms are intended to encompass an assembly of component parts regardless of whether the assembly is oriented in the particular orientation shown in the drawings and described in the specification, upside down from that orientation, or any other rotational variation.

The compositions and methods described herein are illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims.

Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

What is claimed is:

1. A centrifugal apheretic system comprising:
an inlet configured to obtain a biological fluid containing cells;
a collection container configured to collect the biological fluid containing cells from the inlet;
a centrifuge device connected to the collection container, wherein the centrifuge device is configured to rotate about an axis, causing particles in the biological fluid containing cells to separate in the collection container by a particle size and/or a particle flow rate;
an outlet configured to direct a first portion of the separated particles in the biological fluid containing cells from the collection container to a first channel of the outlet and direct a second portion of the separated particles in the biological fluid containing cells from the collection container to a second channel of the outlet.

2. The centrifugal apheretic system of claim 1, further comprising:
a robotic arm connected to the inlet; and
a dual needle connected to the robotic arm, wherein the robotic arm is configured to move the dual needle into the collection container, wherein a first needle of the dual needle is configured to provide the biological fluid containing cells to the collection container, and a second needle of the dual needle is configured to direct the separated particles from the collection container to the outlet.

3. The centrifugal apheretic system of claim 1, wherein the centrifuge device is a center axis centrifuge device configured to rotate about a central axis, and wherein the collection container is disposed at a periphery of the center axis centrifuge device.

4. The centrifugal apheretic system of claim 1, wherein the centrifuge device is a spinning centrifuge device configured to surround the collection container and rotate the collection container about the central axis.

5. The centrifugal apheretic system of claim 1, wherein the centrifuge device is an off-center axis centrifuge device configured to rotate offset relative to the central axis.

6. The centrifugal apheretic system of claim 5, further comprising:
at least two off-center axis centrifuge devices connected to the inlet in parallel, wherein each of the at least two off-center axis centrifuge devices comprise collection containers and each collection container is configured to receive part of the biological fluid containing cells.

7. The centrifugal apheretic system of claim 1, further comprising:
a motorized valve connected to the outlet, wherein the motorized valve is configured to open to either the first channel to direct the first portion of the separated particles to the first channel or the second channel to direct the second portion of the separated particles to the second channel.

8. The centrifugal apheretic system of claim 7, further comprising:
an image sensor connected to the motorized valve and configured to capture an image of the separated particles of the biological fluid containing cells; and
a computing node electrically connected to the sensor, wherein the computing node comprises a processor and a memory, wherein the memory comprises instructions that are configured to cause the processor to:
obtain the image from the image sensor;
derive a parameter of at least a portion of separated particles depicted in the image;
determine whether the separated particles depicted in the image are part of the first portion or the second portion of the biological fluid containing cells based on the derived parameter; and
cause the motorized valve connected to the outlet to open either the first channel or the second channel based on whether the separated particles included in the image are part of the first portion or the second portion of the biological fluid containing cells.

9. The centrifugal apheretic system of claim 8, wherein the biological fluid containing cells comprises a stream of blood cells, and wherein the memory of the computing node comprises instructions that are further configured to cause the processor to:
determine a color of at least the portion of the separated particles depicted in the image; and
derive a hematocrit level of the at least the portion of the separated particles depicted in the image based on the determined color.

10. The centrifugal apheretic system of claim 1, wherein the first channel is configured to direct the first portion of the separated particles to another container for further testing, and the second channel is configured to direct the second portion of the separated particles to a patient via a return line.

11. A method comprising:
obtaining, at an inlet, a biological fluid containing cells, wherein the biological fluid containing cells are directed from the inlet to a collection container connected to a centrifuge device;
causing rotation of the centrifuge device about an axis to separate particles in the biological fluid containing cells in the collection container;
drawing a first portion of the separated particles in the biological fluid containing cells from the collection container to a first channel of the outlet; and
drawing a second portion of the separated particles in the biological fluid containing cells from the collection container to a second channel of the outlet.

12. The method of claim 11, further comprising:
pressurizing, by a pump, the biological fluid containing cells in the inlet, wherein the biological fluid containing cells is obtained at the inlet from a patient, and wherein the second portion of the separated particles in the biological fluid containing cells is directed from the second channel to the patient.

13. The method of claim 11, further comprising:
opening, by a motorized valve connected to the outlet, either the first channel to direct the first portion of the separated particles to the first channel or the second channel to direct the second portion of the separated particles to the second channel.

14. The method of claim 13, further comprising:
capturing, by an image sensor connected to the outlet, an image of the separated particles of the biological fluid containing cells;
obtaining, by a computing node, the image from the image sensor;
deriving, by the computing node, a parameter of at least a portion of separated particles depicted in the image;
determining, by the computing node, whether the separated particles depicted in the image are part of the first portion or the second portion of the biological fluid containing cells based on the derived parameter; and
causing, by the computing node, the motorized valve connected to the outlet to open either the first channel or the second channel based on whether the separated particles included in the image are part of the first portion or the second portion of the biological fluid containing cells.

15. The method of claim 11, further comprising:
moving, by a robotic arm connected to the inlet and to a dual needle, the dual needle into the collection container, wherein a first needle of the dual needle is configured to provide the biological fluid containing cells to the collection container and a second needle of the dual needle is configured to direct the separated particles from the collection container to the outlet.

16. The method of claim 11, further comprising:
disposing, by an injector, an anti-coagulant into the biological fluid containing cells in the inlet.

17. A system comprising:
an inlet configured to obtain a biological fluid containing cells;
a collection container configured to collect the biological fluid containing cells from the inlet;
a centrifuge device connected to the collection container, wherein the centrifuge device is configured to rotate about an axis, causing particles in the biological fluid containing cells to separate in the collection container;
an outlet comprising a first channel and a second channel;
a motorized valve configured to open the first channel and the second channel;
an image sensor connected to the motorized valve and configured to capture an image of the separated particles of the biological fluid containing cells; and
a computing node electrically connected to the sensor, wherein the computing node comprises a processor and a memory, wherein the memory comprises instructions that are configured to cause the processor to:
obtain the image from the image sensor;
derive a particle size of at least a portion of separated particles depicted in the image;
determine whether the separated particles depicted in the image are part of the first portion or the second portion of the biological fluid containing cells based on the derived particle sizes; and
cause the motorized valve connected to the outlet to open either the first channel or the second channel based on whether the separated particles included in the image are part of the first portion or the second portion of the biological fluid containing cells.

18. The system of claim 17, wherein the outlet is configured to direct a first portion of the separated particles in the biological fluid containing cells from the collection container to the first channel of the outlet and direct a second portion of the separated particles in the biological fluid containing cells from the collection container to the second channel of the outlet.

19. The system of claim 17, further comprising:
a robotic arm connected to the inlet; and
a dual needle connected to the robotic arm, wherein the robotic arm is configured to move the dual needle into the collection container, wherein a first needle of the dual needle is configured to provide the biological fluid containing cells to the collection container, and a second needle of the dual needle is configured to direct the separated particles from the collection container to the outlet.

20. The system of claim 17, wherein the centrifuge device is a center axis centrifuge device configured to rotate about a central axis, and wherein the collection container is disposed at a periphery of the center axis centrifuge device.
